(19) United States
(12) Patent Application Publication
(10) Pub. No.: US 2026/0286307 A1
(43) Pub. Date: Sep. 24, 2026

(54) BIOMIMETIC 3D-PRINTED HIERARCHICAL IN VITRO HUMAN SYNOVIUM MODEL FOR ANTI-ARTHRITIS DRUG EVALUATION

(71) Applicant: Center for Neuromusculoskeletal Restorative Medicine Limited, Hong Kong (HK)

(72) Inventors: Dongan WANG, Hong Kong (HK); Huiqun ZHOU, Hong Kong (HK)

(21) Appl. No.: 19/419,336

(22) Filed: Dec. 15, 2025

Related U.S. Application Data
(60) Provisional application No. 63/773,912, filed on Mar. 18, 2025.

(51) Int. Cl. C12N 5/077; B33Y 80/00; C12N 5/0786

(52) U.S. Cl. CPC C12N 5/0652; B33Y 80/00; C12N 5/0645; C12N 2503/02; C12N 2503/04; C12N 2513/00; C12N 2533/50; C12N 2533/54; C12N 2533/90; C12N 2535/10

(57) ABSTRACT

A bio-printed in vitro three-dimensional (3D) human synovium model, comprising: an upper layer comprising human synovial cells and human macrophages exhibiting a pro-inflammatory phenotype; and a lower layer comprising human macrophages exhibiting an anti-inflammatory phenotype; wherein the model is configured to mimic the inflammatory microenvironment of human synovial tissue. The present invention also discloses a method for constructing an in vitro three-dimensional (3D) human synovium model, comprising the steps of preparing hydrogel cell inks; incorporating one or more human cells exhibiting an anti-inflammatory phenotype into the hydrogel to form at least one layer; incorporating one or more human synovial cells and human cells exhibiting a pro-inflammatory phenotype into the hydrogel to form at least one layer above the anti-inflammatory layer; and performing crosslinking and culturing the hydrogel to establish an inflammatory microenvironment, thereby forming an in vitro 3D human synovium model.

### CROSS-REFERENCE TO RELATED APPLICATIONS

Not applicable.

### FIELD OF THE INVENTION

The present invention relates to a human synovium model. More particularly, the present invention pertains to a biomimetic, three-dimensional (3D)-printed, hierarchical in vitro model for evaluating anti-arthritis drugs.

### BACKGROUND OF THE INVENTION

Arthritis is a group of diseases that primarily affect the joints, leading to inflammation, pain and progressive disability. The synovial membrane, which lines the joints, plays a crucial role in arthritis as it becomes inflamed, contributing to joint damage and dysfunction. Inflammatory processes in the synovium are central to many forms of arthritis, such as rheumatoid arthritis (RA). Reference is made to Black, R. J. et al., Global, regional, and national burden of rheumatoid arthritis, 1990-2020, and projections to 2050: a systematic analysis of the Global Burden of Disease Study 2021. The Lancet Rheumatology 2023, 5 (10), e594-e610 discloses that RA is one of the most prevalent forms of arthritic joint disorders, affecting an estimated 18 million people worldwide. To accelerate the discovery of novel therapeutic agents for arthritis, more accurate drug-testing models for efficient and cost-effective drug screening are needed. In vitro 2D cell culture has long been a standard method for studying disease mechanisms and screening potential drug candidates, allowing for controlled experimentation and preliminary assessments of drug efficacy and safety. While 2D cell culture is relatively simple and widely used, it has significant limitations for modeling diseases like arthritis. Reference is made to Johnson, P. A. et al., Three-dimensional, in-vitro approaches for modelling soft-tissue joint diseases. The Lancet Rheumatology 2023 discloses that 2D cultures do not replicate the complex three-dimensional structure of tissues, which can affect cell behavior and drug responses.

In addition to that, researchers utilize a micromass 3D culture in vitro synovium model to evaluate the safety and efficacy of anti-arthritis drugs. Micromass 3D culture is a technique where cells are cultured in a dense, three-dimensional aggregate, which simulates the conditions of in vivo tissues more accurately than traditional 2D cultures. Reference is made to Broeren, M. G. et al., A three-dimensional model to study human synovial pathology. 2019, which discloses that the model recreates the affected synovium seen in RA by co-culturing synovial fibroblasts (key cells in the synovial membrane) and CD14+ mononuclear cells. The cells are embedded in a Matrigel-based micromass, which serves as an extracellular matrix mimic, promoting cell interactions and creating a more realistic environment for studying drug effects. Reference is made to Choudhary, N. et al., Experimental animal models for rheumatoid arthritis. Immunopharmacology and immunotoxicology 2018, 40 (3), 193-200 discloses that animal models, such as collagen-induced arthritis (CIA) or adjuvant-induced arthritis (AIA), are widely used to study the efficacy and safety of drugs in rats, rabbits, and other animals. However, the animal models are limited by high costs, ethical concerns and differences between the model and human arthritis conditions, as cell behaviors vary from species to species, and the cells' sensitivity to drugs also differs.

There are few existing methods and systems in the market that may assist in overcoming the above-mentioned disadvantages.

WIPO patent publication no. 2024073650 discloses a synovium on a chip device having a cartridge housing, a central chamber embedded in the cartridge housing, at least one aperture fluidly connected to the central chamber, and a plurality of evenly spaced micropillars arranged in a substantially circular shape within the central chamber such that the central chamber is partitioned into at least a first inner region and a first outer region, wherein the first inner region includes endothelial cells configured to mimic a vascular network and the first outer region includes fibroblast-like synoviocytes (FLS) configured to mimic a synovium. The device is used in pre-clinical drug testing to support early-stage drug discovery work for treatments for RA and other disorders of the synovium. However, the organ-on-a-chip model disclosed in the said patent is more complex and expensive to create and maintain than traditional in vitro models. This system may require specialized microfluidic devices, advanced equipment, or multi-cellular integration.

United States patent publication no. 20220243157A1 discloses a bioreactor device that mimics a mammalian joint. The 3D human micro-joint chip is physiologically analogous to the native joint and is capable of modeling pathogenesis and treatment of joint diseases, supporting the screening and development of disease-modifying treatments such as medications. The hydrogel in the said patent is a photocrosslinked gelatin hydrogel, which heavily relies on exposure to light and requires specific light wavelengths to active crosslinking agents. Proper safety measures may need to be in place to handle UV light exposure, which can be hazardous to both the researchers and the environment. Variations in light intensity, distance to the light source, and exposure time can all affect the consistency of the crosslinking process.

United States patent publication no. 20210201702A1 discloses a method of generating cellularized ex vivo tissue constructs, comprising: suspending a plurality of viable cells and/or a plurality of tissue fragments in a liquid-phase hydrogel to prepare a composite and/or solution; encapsulating a free-standing evacuable filament structure within the composite and/or solution; gelating the composite and/or solution to mimic an extracellular matrix; and dissolving and evacuating the free-standing filament structure to create at least one lumen in the cellular matrix. However, the 3D-printed freestanding structures are used for ex vivo tissue, which may have a limited lifespan outside the organism. Additionally, using animal-derived tissues or human tissues in ex vivo studies may raise ethical concerns, particularly regarding how the tissue is sourced.

Therefore, there is a need in the art for an in vitro 3D model that includes 3D cultures, allowing cells to interact with each other and with the extracellular matrix (ECM) more naturally, mimicking the tissue architecture found in vivo, and address the drawbacks of animal models and existing in vitro 3D models, while fulfilling the need for efficient drug screening. The described 3D model enables multicellular interactions within an extracellular matrix, thereby mimicking the in vivo tissue organization. This architecture may represent various joint components, facilitating the study of inflammatory processes and drug responses within an arthritis-relevant microenvironment.

### SUMMARY OF THE INVENTION

It is an objective of the present invention to provide a bio-printed in vitro 3D human synovium model of arthritis that offers a high-throughput platform for screening preclinical drugs for anti-arthritis therapeutics.

It is also an objective of the present invention to provide a bio-printed in vitro 3D human synovium model of arthritis, which enables high-throughput testing of potential treatments to significantly reduce the time and resources required.

Accordingly, these objectives may be achieved by following the teachings of the present invention. The present invention relates to a bio-printed in vitro three-dimensional (3D) human synovium model, comprising: an upper layer comprising human synovial cells and human macrophages exhibiting a pro-inflammatory phenotype; and a lower layer comprising human macrophages exhibiting an anti-inflammatory phenotype; wherein the model is configured to mimic the inflammatory microenvironment of human synovial tissue.

### BRIEF DESCRIPTION OF DRAWINGS

The features of the invention will be more readily understood and appreciated from the following detailed description when read in conjunction with the accompanying drawings of the preferred embodiment of the present invention.

FIG. 1A illustrates live or dead staining and CM-H2DCFDA assay of FLS and proinflammatory macrophages within printed hydrogel on Day 7; and FIG. 1B illustrates the invasion of FLS in the established human synovial 3D in vitro model of arthritis.

FIG. 2 illustrates rheological characteristics of Gelatin-Matrigel composite inks; wherein FIG. 2A illustrates the viscosity of different inks at increasing shear rates; and FIG. 2B illustrates the transient, loading, and recovery phases of inks in a thixotropy test.

FIG. 3A-3B illustrate a morphology of printed structures with light microscopy images and printability factor (Pᵣ) of printed constructs.

FIG. 4 illustrates gating strategy and purity of human CD206^{pos}STREM2^{pos} macrophages as determined by flow cytometry cell sorting.

FIG. 5 illustrates design and images of the hierarchical structures of the printed hydrogel.

FIG. 6 illustrates biocompatibility study of printed hydrogel wherein FIG. 6A illustrates Calcein AM staining (stain live cells) of FLS (top) and macrophages (bottom) inside 3D-printed constructs; and FIG. 6B illustrates CCK8 assay results indicating the viability of FLS and macrophages on Day 1 (D1), Day 4 (D4), and Day 7 (D7).

FIG. 7A-7F illustrate establishment of inflammation environment in the human synovial 3D in vitro model of arthritis.

FIG. 8A-8B illustrate drug testing for the established human synovial 3D in vitro model of arthritis.

FIG. 9A illustrates quantitative assessment of the ROS levels in the established human synovial 3D in vitro model of arthritis after drug treatment. FIG. 9B and FIG. 9C illustrate the expression of CD163 and CD86 on cells, as evaluated by flow cytometer.

### DETAILED DESCRIPTION OF THE INVENTION

For the purposes of promoting and understanding of the principles of the invention, reference will now be made to the embodiments illustrated in the drawings and described in the following written specification. It is understood that the present invention includes any alterations and modifications to the illustrated embodiments and includes further applications of the principles of the invention as would normally occur to one skilled in the art to which the invention pertains.

The present invention teaches a bio-printed in vitro three-dimensional (3D) human synovium model, comprising: an upper layer comprising human synovial cells and human macrophages exhibiting a pro-inflammatory phenotype; and a lower layer comprising human macrophages exhibiting an anti-inflammatory phenotype; wherein the model is configured to mimic the inflammatory microenvironment of human synovial tissue.

In accordance with a preferred embodiment of the present invention, the upper layer is stimulated with one or more pro-inflammatory agents, including lipopolysaccharide (LPS), cytokines, or other inflammatory molecules.

In accordance with a preferred embodiment of the present invention, the synovial cells in the upper layer are fibroblast-like synoviocytes (FLS). The FLS are encapsulated with hydrogel ink, which comprises gelatin and Matrigel.

In accordance with a preferred embodiment of the present invention, the gelatin is present in an amount of approximately 4-6% (w/v) in phosphate-buffered saline (PBS) solution and the Matrigel is present in an amount of approximately 10-30% (w/v) in the gelatin solution.

In accordance with a preferred embodiment of the present invention, a transglutaminase (Tgase) is used as a crosslinker and is present in an amount of approximately 0.5-1.5% (w/v) in the gelatin-Matrigel solution.

In accordance with a preferred embodiment of the present invention, the upper layer is cylindrical and arranged in a grid pattern, enclosed by a dish-like dense construct of the lower layer.

In accordance with a preferred embodiment of the present invention, the model comprises two or more layers, each comprising one or more cell types.

In accordance with a preferred embodiment of the present invention, the 3D human synovium model is prepared by 3D printing and generated with a hierarchical printed structure. The hierarchical printed structure is then examined using light microscopy and fluorescence imaging.

In accordance with a preferred embodiment of the present invention, the hierarchical printed structure supports cell survival and proliferation.

The present invention also teaches a method for constructing an in vitro three-dimensional (3D) human synovium model, comprising the steps of: preparing hydrogel cell inks; incorporating one or more human cells exhibiting an anti-inflammatory phenotype into the hydrogel to form at least one layer; incorporating one or more human synovial cells and human cells exhibiting a pro-inflammatory phenotype into the hydrogel to form at least one layer above the anti-inflammatory layer; and performing crosslinking and culturing the hydrogel to establish an inflammatory microenvironment, thereby forming an in vitro 3D human synovium model.

In accordance with a preferred embodiment of the present invention, the human synovial cells comprise fibroblast-like synoviocytes (FLS).

In accordance with a preferred embodiment of the present invention, the human cells exhibiting a pro-inflammatory phenotype are stimulated with one or more pro-inflammatory agents, including lipopolysaccharide (LPS), cytokines, or other inflammatory molecules.

In accordance with a preferred embodiment of the present invention, the method further comprises the steps of: inoculating hydrogel cell inks at a predetermined concentration; and culturing the cells at the predetermined temperature for an extended period. The cells are inoculated in an amount of 5×106-1×107 per millilitre and cultured at a temperature of 36-38° C., and the culture time exceeds 7 days.

In accordance with a preferred embodiment of the present invention, the preparing of hydrogel gel inks comprises the steps of: dissolving gelatin powder in phosphate-buffered saline (PBS) and sterilizing the gelatin solution; sterilizing transglutaminase solution by filtration; simultaneously transferring Matrigel into liquid form; and mixing the gelatin-PBS solution, transglutaminase solution and Matrigel solution.

In accordance with a preferred embodiment of the present invention, the hydrogel layers are incorporated using one or more biofabrication techniques, comprises the steps of: extruding the prepared hydrogel cell inks; and depositing the extruded hydrogel in successive layers to form a three-dimensional structure. The temperatures of the cartridge holder and the needle are set to room temperature (25° C.). The prepared cell ink is extruded through a 0.25 mm needle at a print head speed of 1-3 mm/s under a dosing pressure of 1-5 kPa. All samples are printed in layers with a 0.25 mm layer height. These 3D-bioprinted constructs are then crosslinked for at least 30 min in an incubator at 37° C.

In accordance with a preferred embodiment of the present invention, the establishing of an inflammatory environment is demonstrated by upregulated expression of pro-inflammatory mediators.

In accordance with a preferred embodiment of the present invention, the demonstration by upregulated expression of pro-inflammatory mediators comprises the step of: testing and validating the effects of anti-arthritis drugs selected from a group of celecoxib and tofacitinib. The in vitro synovium model of arthritis in the present invention is used for drug discovery, drug evaluation, preclinical research, toxicity testing, absorption, distribution, metabolism, and excretion testing (ADME), drug metabolism testing and pharmacokinetics (DMPK) and/or disease modelling.

The invention describes a biomimetic human synovial 3D in vitro model of arthritis incorporating human FLS, human pro-inflammatory macrophages and human anti-inflammatory macrophages for protection. The hierarchical structure of synovial lining layer is fabricated using 3D bioprinting. The incorporation of pro-inflammatory macrophages induces the increased production of pro-inflammatory mediators and degradative enzymes, creating an inflammatory microenvironment mimicking arthritis. The inflammatory microenvironment reproduced by the bioprinted synovial construct reflects pathological conditions characteristic of arthritic joints, including processes that influence adjacent tissues through cytokine and enzyme activity. Drug evaluation is carried out via testing responses of the platform to representative anti-arthritis drugs.

Preferably, the cells inside the hydrogel are human primary FLS and human THP-1 derived macrophages. PMA (phorbol 12-myristate-13-acetate)-differentiated human THP-1 cells are treated with LPS to obtain pro-inflammatory macrophages while human THP-1 derived macrophages are treated with IL-4 and IL-13 to obtain anti-inflammatory macrophages. These are subsequently analyzed by flow cytometry to acquire human CD206^{pos}STREM2^{pos} macrophages.

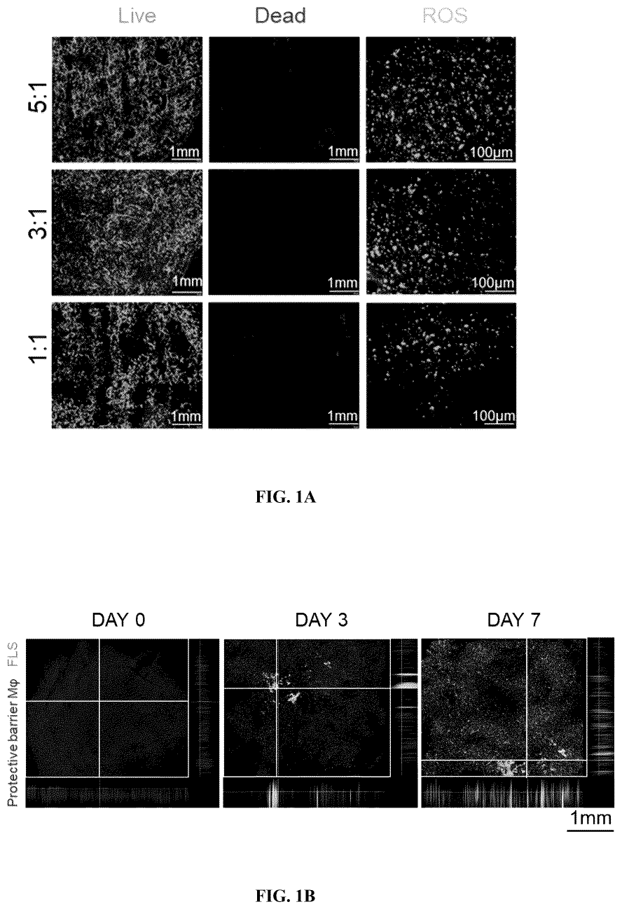

In RA pathogenesis, FLS interact with immune cells to exacerbate inflammation. To further simulate the microenvironment in RA, FLS and proinflammatory macrophages (M1) are coloaded into the bioink to form the top layer of the synovium model. Preferably, the ratio of FLS to pro-inflammatory macrophages in the upper layer ranges from 1:5 to 1:1. The printed hydrogel is cultured in vitro to establish an inflammatory environment and forms an in vitro three-dimensional synovium model for arthritis. The culture temperature is preferably maintained at 36-38° C., with a culture of more than 7 days. The viability and reactive oxygen species (ROS) levels of FLS and M1 macrophages within the printed hydrogel are assessed by conducting a live and dead assay and a CM-H2DCFDA assay on Day 7, as illustrated in FIG. 1A. The results indicate that ROS levels and the number of dead cells increase significantly with higher proportions of proinflammatory macrophages. Accordingly, a ratio of 1:3 is employed due to the reduced number of dead cells and the relatively high ROS levels that resemble in vivo RA conditions. Therefore, in one example, the ratios of FLS to M1 macrophages in the present invention are adjusted to 1:5, 1:3 and 1:1.

### EXAMPLE

To better understand the present invention, the specific content will be further elaborated below in conjunction with the implementation examples. However, the content of this invention is not limited to the following examples. The technical personnel may make various changes or modifications to the invention, and these equivalent forms are also within the limits of the claims listed in this application.

Example 1 describes the preparation of Gelatin-Matrigel composite inks. Gelatin powder is dissolved in PBS and autoclaved for sterilization. The transglutaminase solution (TGase, 20.0% w/v in PBS) is filtered through a 0.25 μm syringe filter for sterilization. The two solutions are then mixed to obtain a final concentration of 5% w/v gelatin, 1% w/v TGase, and are subsequently named 5G.

Example 2 describes the preparation of Gelatin-Matrigel composite inks. Gelatin powder is dissolved in PBS and autoclaved for sterilization. The transglutaminase solution (TGase, 20.0% w/v in PBS) is filtered through a 0.25 μm syringe filter for sterilization. Meanwhile, Matrigel is placed in a 4° C. refrigerator to convert into liquid form. The three solutions are then mixed to obtain a final concentration of 5% w/v gelatin, 1% w/v TGase, and 15% v/v Matrigel, and are subsequently named 5G-15M.

Example 3 describes the preparation of Gelatin-Matrigel composite inks. Gelatin powder is dissolved in PBS and autoclaved for sterilization. The transglutaminase solution (TGase, 20.0% w/v in PBS) is filtered through a 0.25 μm syringe filter for sterilization. Meanwhile, Matrigel is placed in a 4° C. refrigerator to convert into liquid form. The three solutions are then mixed to obtain a final concentration of 5% w/v gelatin, 1% w/v TGase, with 25% v/v Matrigel, and are subsequently named 5G-25M.

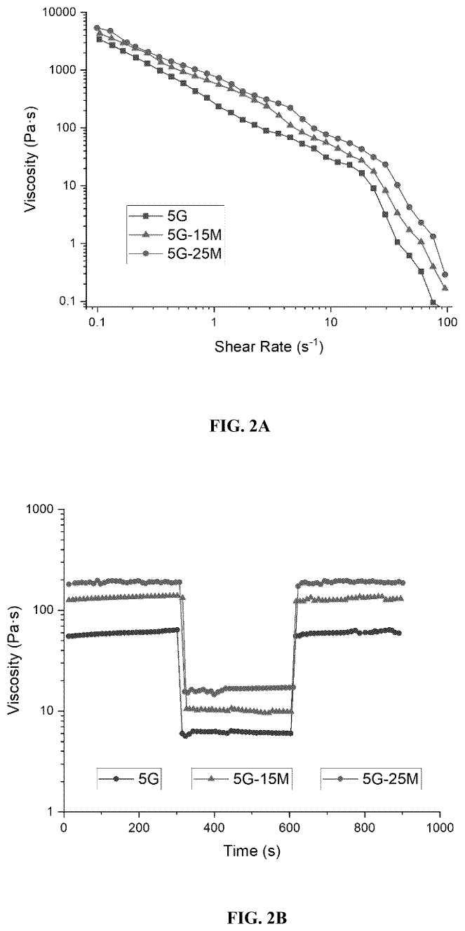

The rheological characteristics of the Gelatin-Matrigel composite ink are measured by steady rate sweeps and cyclic oscillatory shear. Rate sweeps at low strain (1%) are performed to confirm the shear-thinning characteristics of the composite inks over a shear rate range from 0.01 to 100 s⁻¹. To assess the structural recovery of the ink material, thixotropy tests are carried out in three stages: a transient stage (10% strain, lasting 300 s), a loading stage (300% strain, lasting 300 s), and a recovery stage (10% strain, lasting 300 s). All the above tests are conducted at room temperature. The rheological characteristics of the ink are shown in FIG. 2.

As shown in FIG. 2A, the viscosity of the composite inks decreases as the shear rate increases, indicating that all materials exhibit shear-thinning behavior, which is conducive to extrusion-based 3D printing. The shear recovery capability of the bioink materials is assessed through thixotropy testing, demonstrating their ability to preserve structure post-printing as illustrated in FIG. 2B.

Before the bioprinting process, a semi-quantitative assessment is carried out to evaluate the printability and accuracy of the prepared Gelatin-Matrigel ink materials, using the same parameters employed in bioprinting. Optical microscope images of the printed structures are captured for further analysis by Image J. The printability of the bioink materials is determined based on the printability factor (Pr) of the printing structures, defined by the following Equation (1):

$$P r = \frac{π}{4} \times \frac{1}{C} = \frac{{L}^{2}}{16 A} \qquad ( 1 )$$

Where C represents circularity; L denotes the pore perimeter; A is the pore area.

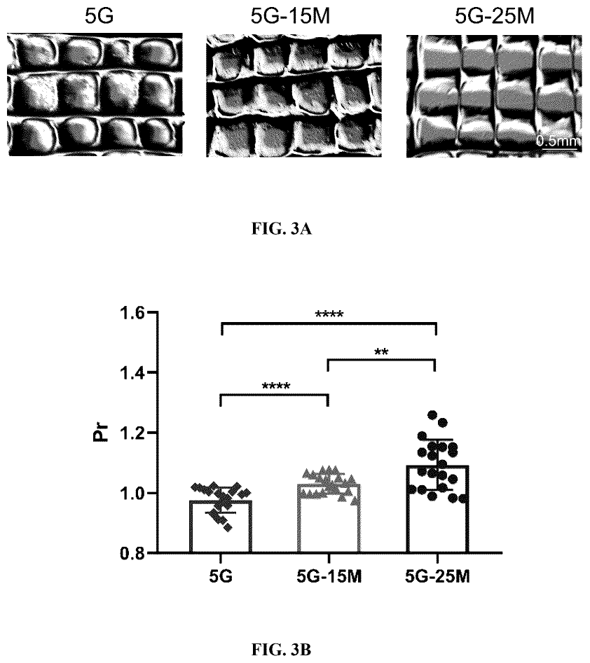

The morphology of printed structures is shown in FIG. 3A-3B. The 5G group exhibited circular pore geometries with a lower Pr factor (Pr=0.97±0.04), showing a significant statistical difference compared to the other two groups. The 5G-25M group (Pr=1.09±0.08) demonstrates reduced printability, while the 5G-15M group (Pr=1.03±0.06) shows optimal printability.

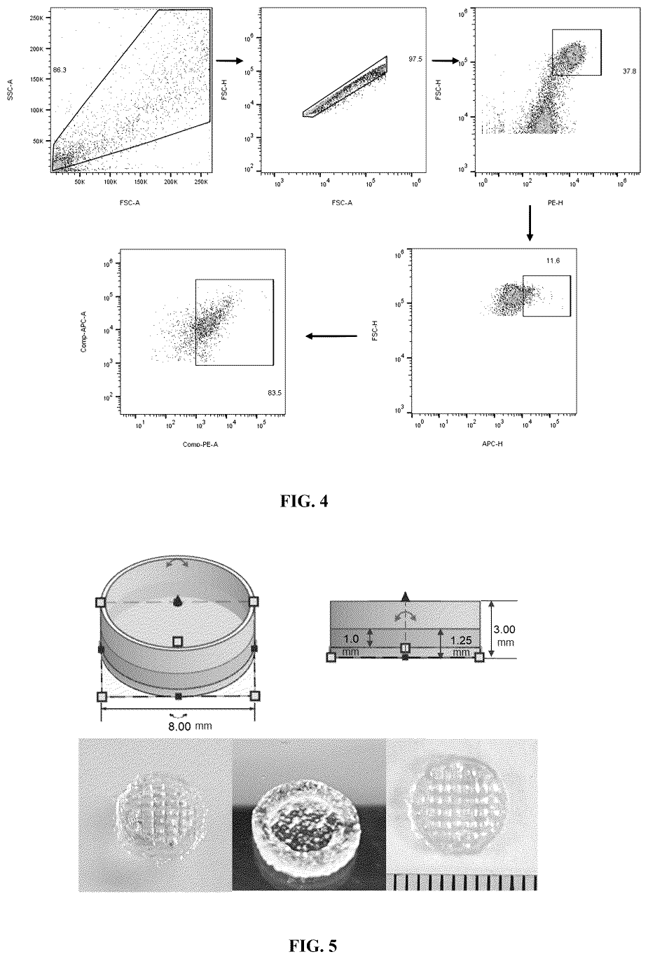

Example 4 describes the preparation of printed hierarchical gelatin-Matrigel hydrogel for constructing an in vitro synovium model of arthritis. Human primary FLS and THP-1 monocytes are used to prepare the bioinks for establishing the model. PMA is employed at a concentration of 100 ng/mL for the differentiation of monocytes into macrophages. To harvest pro-inflammatory macrophages, PMA-differentiated human THP-1 cells are treated with 100 ng/mL of LPS. Meanwhile, anti-inflammatory macrophages are obtained by adding 20 ng/mL of IL-4 and 20 ng/mL of IL-13 to the culture medium for PMA-differentiated THP-1 cells. To acquire human CD206^{pos}STREM2^{pos} macrophages, anti-inflammatory macrophages are collected for flow cytometry sorting. The cells are washed and stained with APC-conjugated human TREM2 antibody and PE-conjugated human CD206 antibody. The gating strategy and purity of isolated cells are shown in FIG. 4.

The prepared composite ink comprising 5% w/v gelatin, 1% w/v TGase and 15% v/v Matrigel with a cell density of 5×10⁶ cells/mL as described in Example 2, is loaded into the printer cartridge for bioprinting. The temperatures of the cartridge holder and the needle are set to room temperature (25° C.). The prepared cell ink is extruded through a 0.25 mm needle with a print head speed of 1.5 mm s⁻¹ and a dosing pressure of 1 kPa. All samples are printed in layers with a 0.25 mm layer height. These 3D-bioprinted constructs are then crosslinked for 30 minutes in an incubator at 37° C. The design of the hierarchical Gelatin-Matrigel hydrogel is a cylindrical structure arranged in a grid pattern, named the pro-inflammatory layer, with overall dimensions of 8×1 millimeters (diameter×height). This is enclosed by a dish-like dense construct, known as protection layer with a height of 3 mm and a bottom thickness of 0.25 mm, as shown in FIG. 5.

3D-printed hierarchical Gelatin-Matrigel hydrogel is further shown in FIG. 5. The two distinct layers (cylinder with grid pattern, the pro-inflammatory layer; and dish-like dense construct, the protection layer) are observed in FIG. 5.

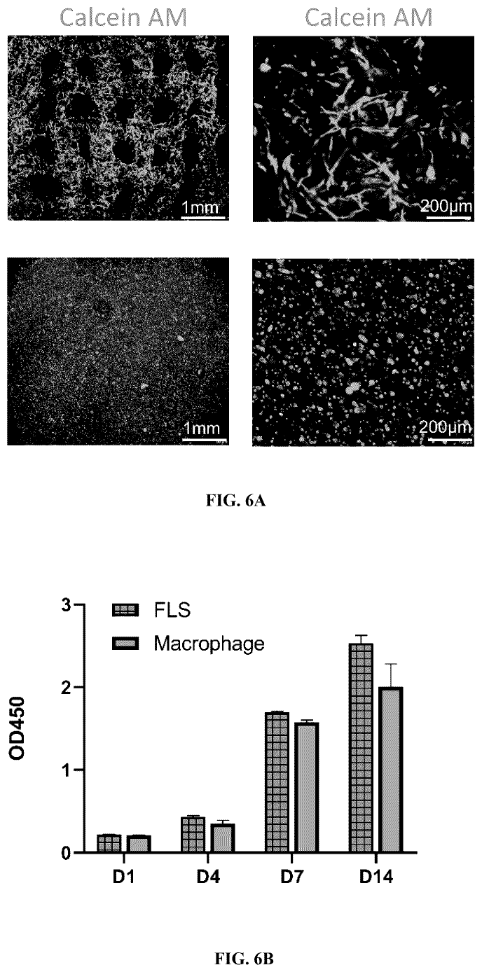

FIG. 6 illustrates biocompatibility study of printed hydrogel. Cell viability of the constructs at Day 7 is assessed using fluorescence staining with Calcein-AM. The printed hydrogel exhibited good cell viability for FLS and macrophages, as shown in FIG. 6A. In addition, cell viability is tested using the CCK8 assay, which is based on the metabolic activity of cells inside the printed constructs. The results demonstrate a gradual increase in cell viability for all groups throughout the entire 7-day culture period, and statistically significant differences are observed when compared to the absorbance at Day 1, as shown in FIG. 6B. These results demonstrate that the composite ink supported cell survival and proliferation without affecting cell viability.

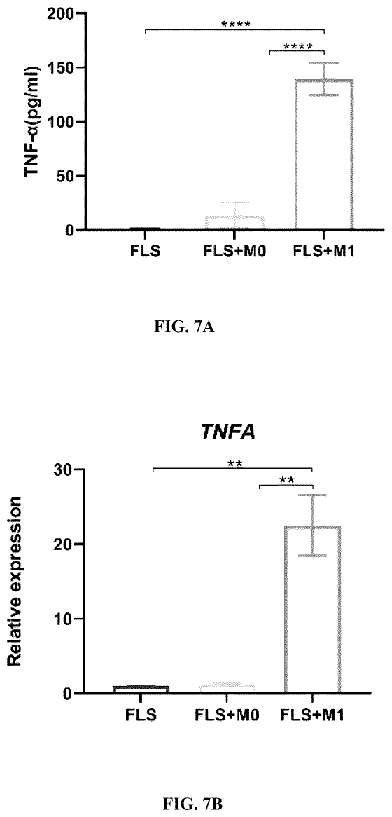

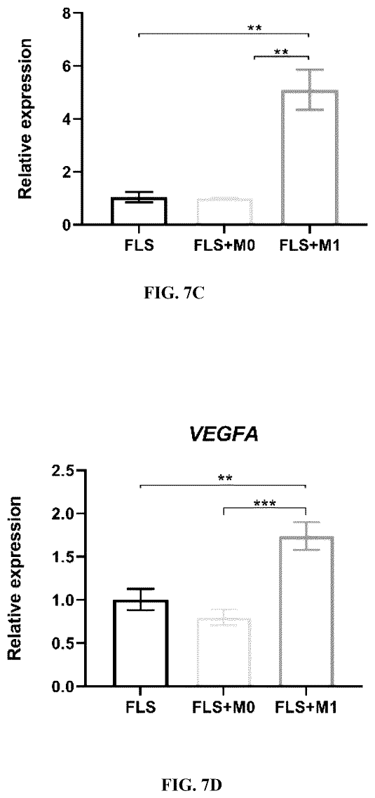

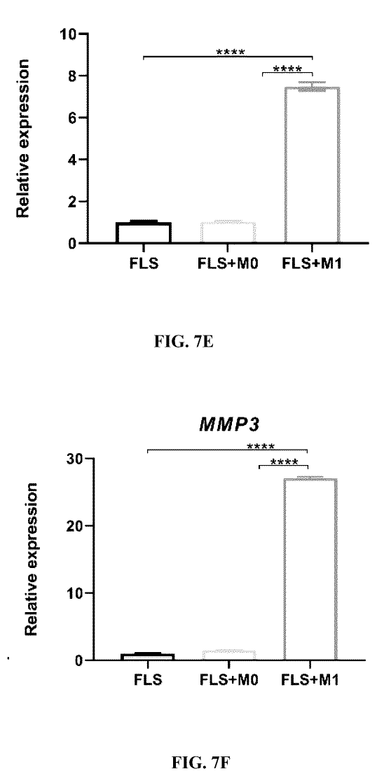

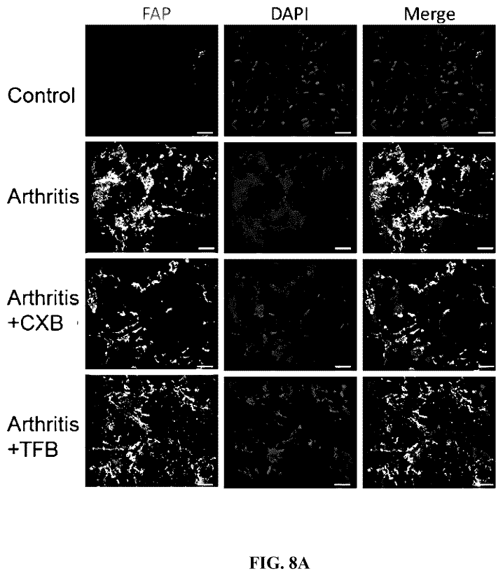

To test the secretion of pro-inflammatory agents at the protein level in the established in vitro model of arthritis on Day 7, the secretion of TNF-α is evaluated by ELISA as shown in FIG. 7A. qRT-PCR is then performed to examine the expression of pro-inflammatory markers (FIG. 7B-7F). TNFA and IL6 are found to be markedly upregulated in the LPS-treated groups (FLS+M1 group) compared to the untreated groups (FLS+M0 group and FLS group). It is known that FLS promote and sustain joint inflammation by generating angiogenic factors (such as VEGF) as arthritis progresses. VEGF expression is observed to be increased in the FLS+M1 group as compared with the FLS+M0 group. Additionally, degradative enzymes such as MMP (matrix metalloproteinases)-1 and MMP-3 are also measured at the transcript level to determine the deconstruction capacity of the developed synovium model of arthritis. Enhanced expression of MMP1 and MMP3 is found in FLS+M1 group.

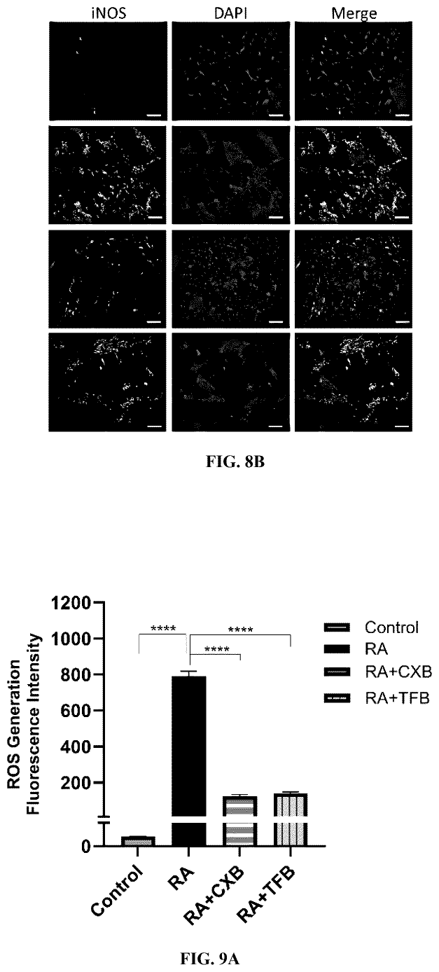

To validate the response of the established model to representative anti-arthritis drugs, such as Celecoxib (CXB) and Tofacitinib (TFB), the expression of fibroblast activation protein alpha (FAP) and inducible nitric oxide synthase (iNOS) in the model is detected through immunofluorescence staining as shown in FIG. 8A-8B. The established synovial model without inflammation induction serves as a control. The results demonstrate that the Arthritis group without drug treatment exhibits the highest expression of FAP and iNOS, indicating the successful establishment of an inflammatory environment in the developed in vitro model of arthritis. Following treatment with CXB and TFB, the samples show attenuated expression of FAP and iNOS, which are associated with the activation and invasion of FLS and the secretion of pro-inflammatory cytokines.

FLS exhibit aggressive characteristics and play a critical role in the invasion and degradation of cartilage during the progression of RA. The invasive growth of FLS and the destruction of the protective barrier layer are analyzed by confocal microscopy using DiO-labelled FLS and DiI-labelled protective barrier macrophages, as illustrated in FIG. 1B. The results demonstrate that the protective layer (black-stained) is gradually disintegrated over time. As inflammation deteriorates, the activated FLS (white-stained) become invasive and infiltrated beneath the protective layer on Day 3 and Day 7.

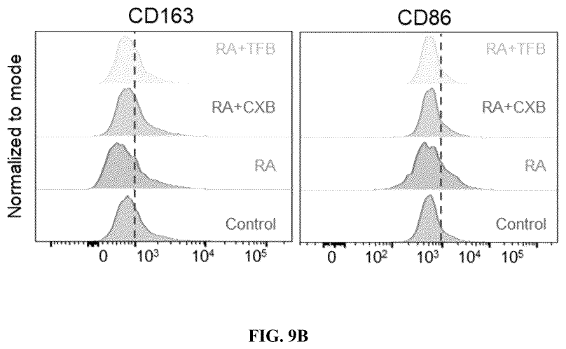

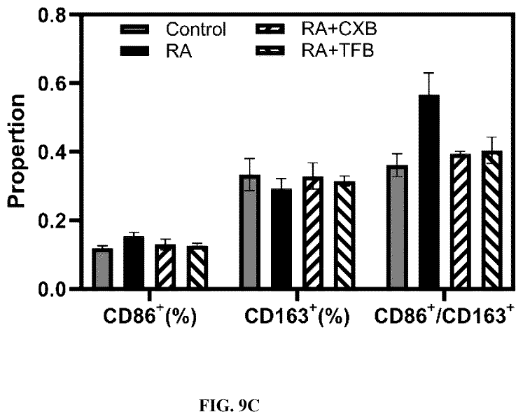

ROS play an important role in the pathogenesis of RA. As illustrated in FIG. 9A, ROS levels are upregulated in the RA group and downregulated following drug treatment in the synovium model. Research has demonstrated the heterogeneity of macrophages in RA pathogenesis and recent evidence indicates that phenotypic changes within macrophage populations are closely associated with RA progression. Accordingly, the functional phenotypes of macrophages in the established RA model with drug treatment are evaluated by flow cytometry, as illustrated in FIGS. 9B and 9C. The RA group exhibits higher expression of CD86, indicating a proinflammatory phenotype on the cell membrane surface, compared to the control and drug-treated groups. Meanwhile, the expression of CD163, which represents an anti-proinflammatory phenotype is reduced in the RA group but upregulated following drug treatment. The ratio of CD86⁺ to CD163⁺ cell proportions is further determined to quantify inflammation levels across healthy, arthritis and remission state. The RA group displayed the highest level of inflammation, demonstrating the successful establishment of an inflammatory environment in the established RA model and its reliable responsiveness to anti-arthritis drugs.

The 3D-printed model in the present invention addresses the drawbacks of animal models and existing in vitro 3D models and fulfils the need for efficient drug screening. It allows for high-throughput testing of potential treatments, significantly reducing the time and resources required compared to the methods mentioned above. The 3D-printed model in the present invention also addresses the challenge of studying the mechanisms of cellular and molecular contributors, as well as the underlying complex interplay of the cells involved, and the impact of potential therapeutic agents in a controlled environment. This mode presents a remarkable proximity to native synovium in the context of arthritis.

The present invention explained above is not limited to the aforementioned embodiment and drawings, and it will be obvious to those having an ordinary skill in the art of the present invention that various replacements, deformations, and changes may be made without departing from the scope of the invention.

1. A bio-printed in vitro three-dimensional (3D) human synovium model, comprising:
an upper layer comprising human synovial cells and human macrophages exhibiting a pro-inflammatory phenotype; and
a lower layer comprising human macrophages exhibiting an anti-inflammatory phenotype;
wherein the model is configured to mimic the inflammatory microenvironment of human synovial tissue.

2. The bio-printed in vitro three-dimensional (3D) human synovium model of claim 1, wherein the upper layer is stimulated with one or more pro-inflammatory agents, including lipopolysaccharide (LPS), cytokines, or other inflammatory molecules.

3. The bio-printed in vitro three-dimensional (3D) human synovium model of claim 1, wherein the synovial cells in the upper layer are fibroblast-like synoviocytes (FLS).

4. The bio-printed in vitro three-dimensional (3D) human synovium model of claim 3, wherein the FLS are encapsulated with hydrogel ink, which comprises gelatin and Matrigel.

5. The bio-printed in vitro three-dimensional (3D) human synovium model of claim 4, wherein the gelatin is present in an amount of approximately 4-6% (w/v) in phosphate-buffered saline (PBS) solution and the Matrigel is present in an amount of approximately 10-30% (w/v) in the gelatin solution.

6. The bio-printed in vitro three-dimensional (3D) human synovium model of claim 5, wherein a transglutaminase (Tgase) is used as a crosslinker and is present in an amount of approximately 0.5-1.5% (w/v) in the gelatin-Matrigel solution.

7. The bio-printed in vitro three-dimensional (3D) human synovium model of claim 1, wherein the upper layer is cylindrical and arranged in a grid pattern, enclosed by a dish-like dense construct of the lower layer.

8. The bio-printed in vitro three-dimensional (3D) human synovium model of claim 1, wherein the model comprises two or more layers, each comprising one or more cell types.

9. The bio-printed in vitro three-dimensional (3D) human synovium model of claim 1, wherein the 3D human synovium model is prepared by 3D printing and generated with a hierarchical printed structure.

10. The bio-printed in vitro three-dimensional (3D) human synovium model of claim 9, wherein the hierarchical printed structure supports cell survival and proliferation.

11. A method for constructing an in vitro three-dimensional (3D) human synovium model, comprising the steps of:
preparing hydrogel cell inks;
incorporating one or more human cells exhibiting an anti-inflammatory phenotype into the hydrogel to form at least one layer;
incorporating one or more human synovial cells and human cells exhibiting a pro-inflammatory phenotype into the hydrogel to form at least one layer above the anti-inflammatory layer; and
performing crosslinking and culturing the hydrogel to establish an inflammatory microenvironment, thereby forming an in vitro 3D human synovium model.

12. The method for constructing an in vitro three-dimensional (3D) human synovium model of claim 11, wherein the human synovial cells comprise fibroblast-like synoviocytes (FLS).

13. The method for constructing an in vitro three-dimensional (3D) human synovium model of claim 11, wherein the human cells exhibiting a pro-inflammatory phenotype are stimulated with one or more pro-inflammatory agents, including lipopolysaccharide (LPS), cytokines, or other inflammatory molecules.

14. The method for constructing an in vitro three-dimensional (3D) human synovium model of claim 11, wherein the method further comprises the steps of:
inoculating hydrogel cell inks at a predetermined concentration; and
culturing the cells at the predetermined temperature for an extended period.

15. The method for constructing an in vitro three-dimensional (3D) human synovium model of claim 11, wherein the preparing of hydrogel gel inks comprises the steps of:
dissolving gelatin powder in phosphate-buffered saline (PBS) and sterilizing the gelatin solution;
sterilizing transglutaminase solution by filtration;
simultaneously transferring Matrigel into liquid form; and
mixing the gelatin-PBS solution, transglutaminase solution and Matrigel solution.

16. The method for constructing an in vitro three-dimensional (3D) human synovium model of claim 11, wherein the hydrogel layers are incorporated using one or more biofabrication techniques, comprises the steps of:
extruding the prepared hydrogel cell inks; and
depositing the extruded hydrogel in successive layers to form a three-dimensional structure.

17. The method for constructing an in vitro three-dimensional (3D) human synovium model of claim 11, wherein the establishing of an inflammatory environment is demonstrated by upregulated expression of pro-inflammatory mediators.

18. The method for constructing an in vitro three-dimensional (3D) human synovium model of claim 17, wherein the demonstration by upregulated expression of pro-inflammatory mediators comprises the step of:
testing and validating the effects of anti-arthritis drugs selected from a group of celecoxib and tofacitinib.
